(19) United States
(12) Patent Application Publication
(10) Pub. No.: US 2005/0125006 A1
(43) Pub. Date: Jun. 9, 2005

(54) Device for sealing a cervical canal

(76) Inventor: Nady E. Nady, Skillman, NJ (US)

Correspondence Address: Gregory C. Houghton, Esq.;Mathews, Collins, Shepherd & McKay, P.A., Suite 306, 100 Thanet Circle, Princeton, NJ, 08540, US

(21) Appl. No.: 10/731,342

(22) Filed: Dec. 9, 2003

(51) Int. Cl. A61B 17/42

(52) U.S. Cl. 606/119

(57) ABSTRACT

A device useful in forming a seal between a hysteroscope instrument and a cervical canal during diagnostic or surgical procedures known as hysteroscopy. The device grasps the exterior of the cervical canal pressing the cervical canal inwardly toward the outer surface of the hysteroscope instrument forming a seal between the outer surface of the hysteroscope instrument and the cervical canal, thereby preventing fluid or gas from exiting the cervical canal.

### BACKGROUND OF THE INVENTION

1. Field of the Invention

The present invention relates to a device useful in forming a seal between a hysteroscope instrument and a cervical canal during diagnostic or surgical procedures known as hysteroscopy. The device grasps the exterior of the cervical canal pressing the cervical canal inwardly toward the outer surface of the hysteroscope instrument forming a seal between the outer surface of the hysteroscope instrument and the cervical canal, thereby preventing fluid or gas from exiting the cervical canal.

2. Related Art

Procedures which require entry into the uterus are well known. Two such procedures are diagnostic and surgical hysteroscopy.

Diagnostic hysteroscopy is a procedure used to help diagnose abnormal uterine bleeding, infertility, uterine and cervical cancer, location of intra uterine devices, complicated abortion and fetal examination. Diagnostic hysteroscopy involves inserting a hysteroscope instrument, which is a thin telescope, through the cervix into the uterus where the endometrial cavity (inside the uterus) can be observed.

Surgical hysteroscopy is a procedure used to remove polyps, cut adhesions, and other surgical procedures. Surgical hysteroscopy employs a type of hysteroscope instrument having channels through in which it is possible to insert very thin instruments to allow surgical procedures to occur within the uterus.

Because the inside of the uterus is a potential cavity, like a collapsed air dome, it is necessary to fill (distend) the uterus with either a fluid or a gas in order to visualize the interior of the uterus during both diagnostic and surgical hysteroscopy. However, the fluid or gas received in the uterus during either diagnostic or surgical hysteroscopy can subsequently flow from the uterus through the cervical canal resulting in a loss of pressure and an incapability of determining the amount of fluid or gas used in the procedure.

It is therefore desirable to provide a device to effectively seal the area between the outer surface of the hysteroscope instrument and the cervical canal, thereby preventing backflow of fluid or gas used to pressurize the uterus during either diagnostic or surgical hysteroscopy.

### SUMMARY OF THE INVENTION

The present invention provides a device for gripping the exterior of a cervical canal to press the cervical canal inwardly against a tubular portion of a hysteroscope instrument inserted through the cervical canal to resist backflow of fluid or gas used as a distension media within a uterus from flowing out of the cervical canal during the use of the hysteroscope instrument. The device includes a pair of pivotally connected cross arms having forward end portions with curved clamping tips and rearward end portions with hand-grips. The connection between the cross arms permits pivotal movement between the cross arms and permits a predetermined longitudinal relative shifting of the cross arms. The curved clamping tips are aligned at an angle relative to the hand-grips and the curved clamping tips define generally a circular area, whereby the clamping tips grip the exterior of the cervical canal to press the cervical canal inwardly against the tubular portion of the hysteroscope instrument.

Also, the device can be locked with a locking device connected to the cross arms wherein the locking device holds the cross arms in a closed position.

Another embodiment of the present invention provides a device for gripping an exterior of a cervical canal to press the cervical canal inwardly against a tubular portion of a hysteroscope instrument inserted through the cervical canal to resist backflow of fluid or gas used as a distension media within a uterus from flowing out of the cervical canal during the use of the hysteroscope instrument. The device includes a flexible band having a forward end portion with a moveable end and a rearward end portion with a fixed end. The moveable end is adapted to receive the cervical canal therebetween and grip the exterior of the cervical canal to press the cervical canal inwardly against the tubular portion of the hysteroscope instrument.

Also, the device can be held or locked with an adjustment means connected to the fixed end wherein holding the moveable end of the device in a fixed position.

### BRIEF DESCRIPTION OF THE DRAWINGS

The advantage, nature, and various additional features of the invention will appear more fully upon consideration of the illustrative embodiments now to be described in detail in connection with accompanying drawings wherein:

FIG. 1 is a schematic diagram of a device for sealing a cervical canal according to an exemplary embodiment of the invention.

FIG. 2 is a schematic diagram of the device of the present invention used when a hysteroscope instrument is inserted into a cervical canal of a subject uterus shown in a partial cross-section.

It should be understood that the drawings are for purposes of illustrating the concept of the invention and are not necessarily to scale.

### DETAILED DESCRIPTION OF THE INVENTION

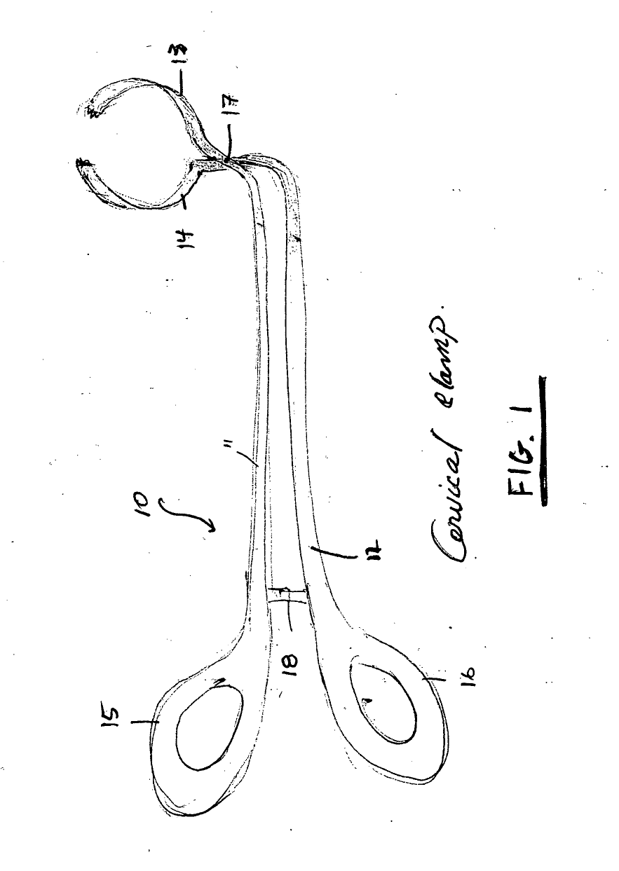

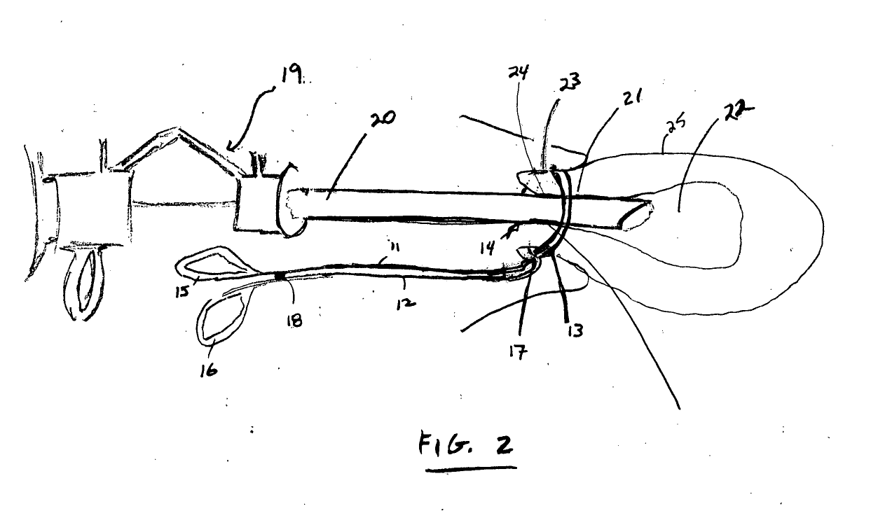

Referring to the drawings wherein like reference numerals identify similar or like elements throughout the several views and initially to FIG. 1 there is shown the present invention, device 10. Device 10 includes cross arms 11 and 12 which are of narrow elongate construction. Cross arms 11 and 12 have curved clamping tips 13 and 14 at one end and hand-grips 15 and 16 at the opposite end. While the curved clamping tips 13 and 14 are shown with a generally circular shape an oval and other curved shapes can also be used. Curved clamping tips 13 and 14 are aligned at an angle relative to hand-grips 15 and 16. The angle of alignment while shown at approximately 90 degrees can range from approximately 45 degrees to approximately 135 degrees.

Cross arms 11 and 12 are pivotally connected to each other with connection 17. The closing of hand-grips 15 and 16 (i.e. bringing hand-grip 15 toward hand-grip 16) enables curved clamping tips 13 and 14 to be brought toward one another and close. Connection 17 can be a pivot point wherein two cross arms are connected and is well understood by those skilled in the art. For example, instruments that have pivotally connected cross arms include scissors, pliers, and hemostats.

Cross arms 11 and 12 can be locked to one another by locking device 18. Locking device 18 holds cross arms 11 and 12 in a closed profile incrementally over an angular range of pivotal movement of cross arms 11 and 12. One exemplary embodiment of locking device 18 is a locking ear system used in hemostats. Other locking devices which are known to those skilled in the art are equally suitable which include but are not limited to a latch-type system used by pliers and/or clippers.

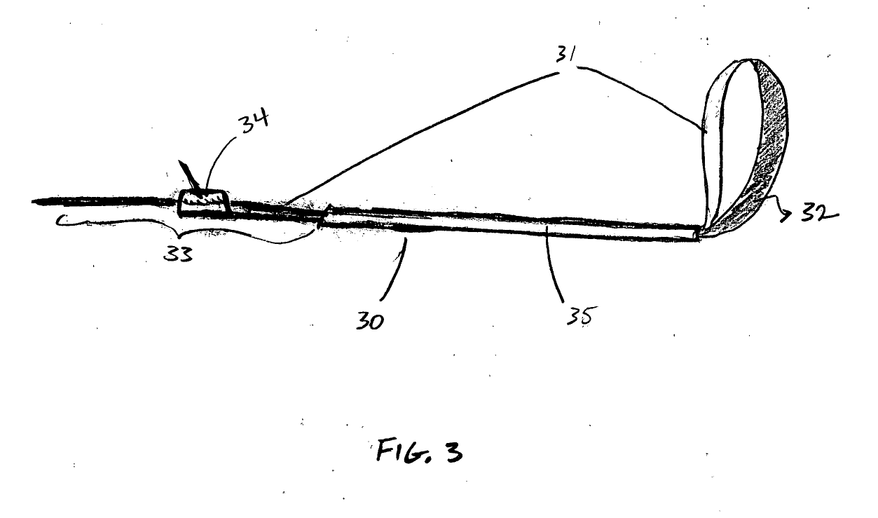

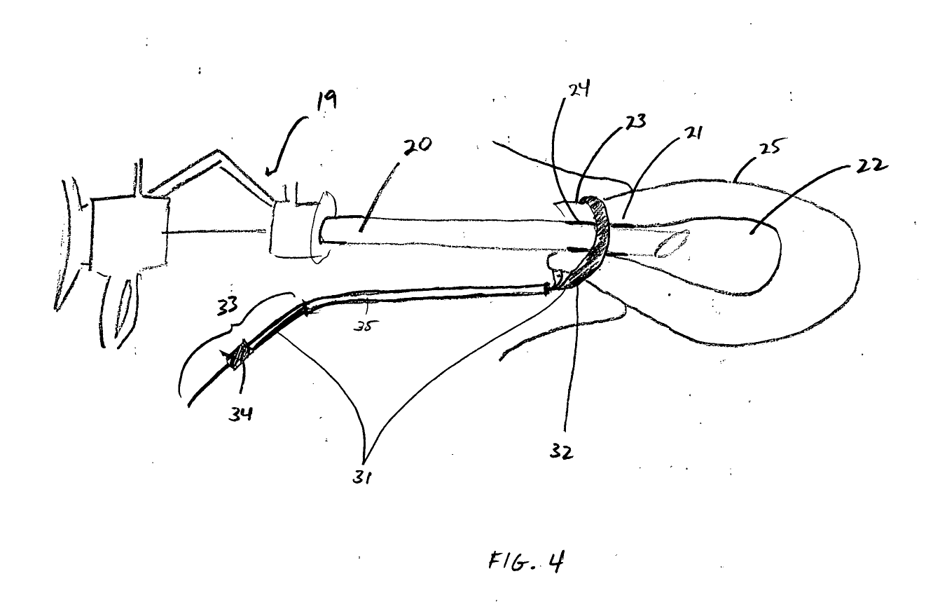

FIG. 2 schematically illustrates device 10 during use of hysteroscope instrument 19. Curved clamping tips 13 and 14 are positioned around the exterior of cervical canal 23. Cross arms 11 and 12 are pivotally moved into a closed position bringing curved clamping tips 13 and 14 toward each other enabling curved clamping tips 13 and 14 to grip exterior 25 of cervical canal 23, inwardly press interior 21 of cervical canal 23 against tubular portion 20 of hysteroscope instrument 19, and form seal 24 between tubular portion 20 of hysteroscope instrument 19 and interior 21 of cervical canal 23. Seal 24 prevents backflow of fluid or gas from uterine cavity 22 through the cervical canal 23.

Curved clamping tips 13 and 14 can be formed of metal or plastic. Curved clamping tips 13 and 14 can be covered, such as with an elastomeric material, for example polyurethane, poly(vinyl chloride) or any other suitable material. The elastomeric material provides comfort and protects the exterior surface of cervical canal 23. The elastomeric material can be replaceable and disposable.

Referring to FIG. 3, there is shown a second embodiment of the invention, device 30. Device 30 includes flexible band 31. Flexible band 31 has a forward end portion with moveable end 32 at one end and a rearward end portion with fixed end 33 at the opposite end. While moveable end 32 is shown with a generally circular shape, an oval and other curved shapes can also be used. Moveable end 32 is aligned at an angle relative to fixed end 33. The angle of alignment while shown at approximately 90 degrees can range from approximately 45 degrees to approximately 135 degrees.

Flexible band 31 can be adjusted by adjustment means 34. Adjustment means 34 changes size of generally circular shape of moveable end 32. Adjustment means 34 can hold and/or lock moveable end 32 in a fixed profile or position. Adjustment means are well known to those skilled in the art and include but are not limited to a latch-type system used by pliers, clippers, ratchets, and/or plastic ties.

Device 30 can be covered with a shield 35. Shield 35 can be formed of an elastomeric material, for example polyurethane, poly(vinyl chloride) or any other suitable material.

FIG. 4 schematically illustrates device 30 during use of hysteroscope instrument 19. Moveable end 32 is positioned around the exterior of the cervical canal 23. Flexible band 31 is adjusted enabling moveable end 32 to grip exterior 25 of cervical canal 23, inwardly press interior 21 of cervical canal 23 against tubular portion 20 of hysteroscope instrument 19 and interior 21 of cervical canal 23. Seal 24 prevents backflow of fluid or gas from uterus cavity 22 through the cervical canal 23.

Moveable end 32 can be formed of an elastomeric material. The elastomeric material provides comfort and protects the exterior surface of the cervical canal.

Device 30 can be replaceable or disposable.

The terms fluid or gas include but are not limited to water, saline, isotonic solution, glycine solutions, carbon dioxide, or any other fluid or gas that would be appreciated by those skilled in the art as to be employed in hysteroscopy.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

1. A device for gripping an exterior of a cervical canal to press the cervical canal inwardly against a tubular portion of a hysteroscope instrument inserted through the cervical canal to resist backflow of fluid or gas used as a distension media within a uterus from flowing out of the cervical canal during the use of the hysteroscope instrument, the device comprising:
a pair of pivotally connected cross arms having forward end portions with curved clamping tips and rearward end portions with hand-grips, the connection between said cross arms permitting pivotal movement therebetween and a predetermined longitudinal relative shifting of said cross arms, said curved clamping tips adapted to receive the cervical canal therebetween and grip the exterior of the cervical canal to press the cervical canal inwardly against the tubular portion of the hysteroscope instrument.

2. The device according to claim 1 wherein said curved clamping tips are aligned at an angle relative to said hand-grips.

3. The device according to claim 2 wherein said hand-grips are approximately perpendicular to said end portion.

4. The device according to claim 2 wherein said angle is approximately between 45 degrees and 135 degrees.

5. The device according to claim 1 further comprising a locking device connected to said cross arms wherein said locking device holds said cross arms in a closed position.

6. The locking device according to claim 5 wherein said locking device holds said cross arms in a closed profile incrementally over an angular range of pivotal movement of said arms.

7. The device according to claim 1 wherein said curved clamping tips define generally a circular area.

8. The device according to claim 1 further comprising a material covering said curved clamping tips.

9. The device according to claim 8 wherein said material is made of an elastomeric material.

10. The material according to claim 8 wherein said material is replaceable.

11. A method of preventing fluid or gas from flowing out of a cervical canal from a uterus during the use of a hysteroscope instrument, the method comprising the steps of:
inserting the hysteroscope instrument into the cervical canal of the uterus;
positioning a device to grip exterior of the cervical canal wherein said device comprises a pair of pivotally connected cross arms having forward end portions with curved clamping tips and rearward end portions with hand-grips, the connection between said cross arms permitting pivotal movement therebetween and a predetermined longitudinal relative shifting of said cross arms, said curved clamping tips adapted to receive the cervical canal therebetween; and
gripping the exterior of the cervical canal with said curved clamping tips to press the cervical canal inwardly against a tubular portion of the hysteroscope instrument for preventing the fluid or gas from the uterus from flowing out of the cervical canal during the use of said hysteroscope instrument.

12. The method of claim 1 1, wherein said curved clamping tips are approximately perpendicular to said hand-grips.

13. The method of claim 11, wherein said device further comprises an elastomeric material covering said curved clamping tips.

14. A device for gripping an exterior of a cervical canal to press the cervical canal inwardly against a tubular portion of a hysteroscope instrument inserted through the cervical canal to resist backflow of fluid or gas used as a distension media within a uterus from flowing out of the cervical canal during the use of the hysteroscope instrument, the device comprising:
a flexible band having a forward end portion with a moveable end and a rearward end portion with a fixed end, said moveable end adapted to receive the cervical canal therebetween and grip the exterior of the cervical canal to press the cervical canal inwardly against the tubular portion of the hysteroscope instrument.

15. The device according to claim 14 wherein said moveable end is approximately perpendicular to said fixed end.

16. The device according to claim 14 further comprising an adjustment means connected to said fixed end wherein said adjustment means holds said moveable end in a fixed position.

17. The device according to claim 14 wherein said flexible band is covered with a shield.

18. The device of claim 14 wherein said device is made of an elastomeric material.

19. The device of claim 18 wherein said device is disposable.

20. A method of preventing fluid or gas from flowing out of a cervical canal from a uterus during the use of a hysteroscope instrument, the method comprising the steps of:
positioning a device to grip exterior of the cervical canal wherein said device comprises a flexible band having a forward end portion with a moveable end and a rearward end portion with a fixed end, said moveable end adapted to receive the cervical canal therebetween;
inserting the hysteroscope instrument into the cervical canal of the uterus; and
gripping the exterior of the cervical canal with said moveable end to press the cervical canal inwardly against a tubular portion of the hysteroscope instrument for preventing the fluid or gas from the uterus from flowing out of the cervical canal during the use of said hysteroscope instrument.

21. The method according to claim 20 wherein said device further comprises an adjustment means connected to said fixed end.

22. The method of claim 21 wherein said adjustment means holds said moveable end in a fixed position.
